(19) United States
(12) Patent Application Publication
(10) Pub. No.: US 2004/0024000 A1
(43) Pub. Date: Feb. 5, 2004

(54) Dihydropyrimidine derivatives as cysteine protease inhibitors

(76) Inventors: Rajeshwar Singh, Alberta (CA); Andhe V.N. Reddy, Alberta (CA); Nian E. Zhou, Napperville (IL); Qizhu Ding, Alberta (CA); George Thomas, Alberta (CA); Jadwiga Kaleta, Alberta (CA); Ronald G. Micetich, Alberta (CA)

Correspondence Address: ROTHWELL, FIGG, ERNST & MANBECK, P.C., 1425 K STREET, N.W., SUITE 800, WASHINGTON, DC, 20005, US

(21) Appl. No.: 10/398,938

(22) PCT Filed: Apr. 30, 2001
(86) PCT No.: PCT/IB01/00707

(51) Int. Cl. A61K 31/513; C07D 239/02

(52) U.S. Cl. 514/269; 544/319

(57) ABSTRACT

Ditiydropyrimidine derivatives are disclosed, which can be used to inhibit cysteine protease activity.

This invention relates to novel derivatives of dihydropyrimidine, to pharmaceutical compositions containing such compounds, and to their use in medicine as inhibitors of lysosomal cysteine proteases, particularly the cathepsins and more particularly Cathepsins B, L, K and S.

### BACKGROUND OF THE INVENTION

Cysteine proteinases contain a highly reactive cysteine sulfhydryl group and a histidine imidazole group within the active site of the enzyme and are known to play an important role in a number of disease states.

Cathepsin K can be secreted into the extracellular space and is involved in bone and cartilage remodelling. Cathepsin K is implicated in the pathogenesis of osteoporosis. Cathepsin K inhibitors can prevent osteoporosis in animal models (PNAS. 1997. 94:14249-14254). Cathepsin L inhibitors have also been shown to inhibit osteoporosis (Bone, 1997. 20:465-471).

Cathepsin B and others have also been shown to be released extracellularly by various tumour cells and are thought to play a role in tumour invasion (Journal of cellular Physiology. 1992.150:534-544).

The cathepsins have also been shown to play a role in rheumatoid arthritis (Arthritis and Rheumatism 1994. 37:236-247) and neuronal and cardiac ischaemia (European Journal of Neuroscience. 1998.10.1723-1733).

Cathepsins S and L both play a role in the generation of free MHC class II molecules capable of binding antigenic peptides in the endosomes. These class II/peptide complexes move to the cell membrane and are involved in T lymphocyte activation. Inhibitors of Cathepsin S have been shown to inhibit allergic immune responses (Journal of Clinical Investigation. 1998.101:2351-2363).

In addition to their role in the above diseases, cathepsins play a major role in the pathogenesis of infectious diseases. For example, cathepsins are used by the protozoal parasites Plasmodium (malaria) and Trypanosoma (Chagas Disease) to invade the human host and cathepsin inhibitors can inhibit experimental disease in both cases (Antimicrobial agents and chemotherapy. 1998. 42:2254-2258; Journal of Experimental Medicine. 1998. 188:725-734). Cysteine proteases are also virulence factors for some pathogenic bacteria (J. Biochem. 1998, 123:305-312, Biochimica et Biophysica Acta 2000,1477:35-50).

Cysteine proteinase are inhibited by several types of peptide derived inhibitors such as peptidyl aldehydes (Eur. J. Biochem. 1982, 129, 33-41), chloromethyl ketones (Acta. Biol. Med. Ger. 1981, 40, 1503-1511), diazomethyl ketones (Biochemistry 1977,16, 5857-5861), monofluoromethyl ketones (Biochemical Pharmacology 1992 44, 1201-1207), acyloxy methyl ketones (J. Med. Chem. 1994, 37, 1833-1840), O-acyl hydroxamates (Biochem. Biophy. Research Communications 1988, 155, 1201-1206), methyl sulphonium salts (J. Biol. Chem. 1988, 263, 2768-2772), epoxy succinyl derivatives (Agric. Biol. Chem. 1978, 42, 523-527), tetrahydrofuran-3-one (WO-50533, 1998), monobactams (U.S. Pat. No. 5,986,108, 1999; U.S. Pat. No. 5,916,887, 1999; U.S. Pat. No. 5,959,123, 1999) and oxapenams (U.S. Pat. No. 5,905,076, 1999; U.S. Pat. No. 5,925,633, 1999) without significantly inhibiting other classes of proteinases.

These inhibitors, in general, have a natural peptidyl affinity group and a reactive group towards the thiol of the cysteine residue of cysteine proteinase. Natural peptidyl affinity groups are an attractive starting point for drug discovery because they are essential to virtually every biochemical process. Unfortunately, the in vivo effectiveness of such compounds is not reflected as expected on the basis of in vitro inhibitory activity, which may be due to the specificity towards other proteinases and poor pharmacokinetics. However, there exists a continuing need to develop new cysteine proteinase inhibitors with high selectivity and lower toxicity.

In an effort to find more effective low molecular weight cysteine protease inhibitors for therapeutic uses, we have focused our attention on a novel dihydropyrimidine class of compounds having substitutions at positions 2,3 and 5 and inhibitors of cysteine proteinase particularly cathepsins, which is reported in the present invention. The novel route using appropriately substituted monobactams as starting material for synthesis of these compounds is also described.

### SUMMARY OF THE INVENTION

The present invention provides the certain derivatives of novel dihydropyrimidine, to pharmaceutical compositions containing such compounds, and to their use in medicine as inhibitors of lysosomal cysteine proteases, particularly the cathepsins and more particularly Cathepsins B, L, K and S.

In accordance with the present invention, there is provided novel dihydropyrimidine derivatives having the formula (I):

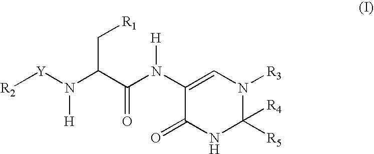

or the pharmaceutically acceptable salts, hydrate or solvate thereof.

The present invention makes available a new class of cysteine protease inhibitors, which are significantly different from those, reported earlier and with improved in vivo potency in laboratory rodents. These compounds are useful for the treatment of diseases mediated by cysteine protease activity, for example muscular dystrophy, osteoporosis, tumour metastasis, rheumatoid arthritis, neuronal or cardiac ischaemia, allergic immune response, and protozoal or bacterial disease.

### DETAILED DESCRIPTION OF THE INVENTION

In accordance with the present invention, there is provided dihydropyrimidine derivatives of general formula (I):

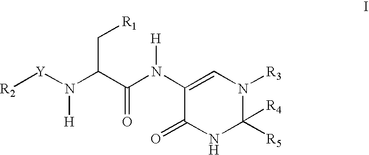

or a pharmaceutical acceptable salt, hydrate or solvate thereof.

Wherein:

Y represents —C(O)—, —OC(O)—, —NHC(O)— or —S(O₂)—;

R₁ represents hydrogen or an optionally substituted C₁-C₆alkyl, C₂-C₆alkenyl, C₂-C₆alkynyl, cycloalkyl, cycloalkenyl, aryl or heterocyclic group.

R₂ represents hydrogen or an optionally substituted C₁-C₆alkyl, C₂-C₆alkenyl, C₂-C₆alkynyl, cycloalkyl, cycloalkenyl, aryl or heterocyclic group.

R₃ represents H, R₆ and OR₆, wherein R₆ is C₁-C₃alkyl, C₂-C₃alkenyl, C₂-C₃alkynyl, cycloalkyl, cycloalkenyl, aryl or a heterocyclic group.

R₄ and R₅ individually represent H or an optionally substituted C₁-C₆alkyl, C₂-C₆alkenyl, C₂-C₆alkynyl, cycloalkyl, cycloalkenyl, aryl, or heterocyclic group.

R₄ and R₅ together represents an oxo group or a C₃-C₆ cyclic ring system, which may be further, substituted with hydroxyl, halogen, and amino and substituted amino groups.

The derivative of formula I having asymmetric carbon atoms represents both R and S diastereoisomers.

The derivative of formula I having double bonds represents both E and Z geometrical isomers.

Pharmaceutically acceptable salts of the compounds of this invention include the sodium, potassium, magnesium, calcium, hydrogen chloride, tartaric acid, succinic acid, fumaric acid, methanesulfonic acid and p-toluenesulfonic acid salts.

As used herein the term “(C₁-C₆) alkyl” or “lower alkyl” means a straight or branched chain alkyl moiety having from 1 to 6 carbon atoms, including for example, methyl, ethyl, propyl, 1-methylethyl, butyl, 1-methylpropyl, 2-methylprop-1-yl, 2-methylprop-2-yl, pentyl, 3-methylbutyl, and hexyl. Similar terms such as “(C₁-C₃) alkyl” are to be interpreted similarly.

As used herein the term “C₂-C₆alkenyl” means a straight or branched chain alkenyl moiety having from 2 to 6 carbon atoms having at least one double bond of either E or Z stereochemistry where applicable. The term includes, for example, vinyl, allyl, 1- and 2-butenyl and 2-methyl-2-propenyl. Similar terms such as “(C₂-C₃)alkenyl” are to be interpreted similarly.

As used herein the term “C₂-C₆ alkynyl” means a straight chain or branched chain hydrocarbon groups having from two to six carbon atoms and having in addition one triple bond. This term would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl. Similar terms such as “(C₂-C₃)alkynyl” are to be interpreted similarly.

As used herein the term “cycloalkyl” means a saturated alicyclic moiety having from 3-7 carbon atoms and includes, for example, cyclohexyl, cycloheptyl, cyclopentyl, cyclobutyl and cyclopropyl.

As used herein the term “halogen” means fluoro, chloro, bromo or iodo.

As used herein the term “aryl” refers to a mono-, bi- or tri-cyclic, substituted or unsubstituted, carbocyclic aromatic group, and to groups consisting of two covalently linked substituted or unsubstituted monocyclic carbocyclic aromatic groups. Illustrative of such groups is phenyl, biphenyl and napthyl, tetrahydronaphthyl, dihydronaphthyl, and cyclohexyl phenyl.

As used herein the unqualified term “heterocyclic” means a 5-7 membered heterocyclic ring, which may be aromatic or non-aromatic, containing one or more heteroatoms selected from S, N and O, and optionally fused to a benzene or hetero-atom containing ring. The term therefore includes C₁-C₁₁ heterocyclic groups containing 1-4 heteroatoms selected from N, S or O. Examples include 1,2,3-triazolyl, 1,2,4-triazolyl, 1,2,3,4-tetrazolyl, thienyl, furyl, pyrrolyl, imidazolyl, pyridyl, pyrimidinyl, oxazolyl, thiazolyl, thiadiazolyl, pyrazolyl, pyrrolidinyl, morpholinyl, thiomorpholinyl, piperazinyl, piperidinyl, benzofuranyl, benzothiophenyl, benzimidazolyl, quinolinyl, isoquinolinyl, indolyl, tetrahydroquinolinyl, tetrahydroisoquinolinyl, pyridylphenyl and pyrimidylphenyl groups.

As used herein, the unqualified term “substituted” as applied to a group means substituted with 1, 2, or 3 substituents selected from

(C₁-C₃)alkyl;

phenyl;

C₃-C₆cycloalkyl;

heterocyclic;

hydroxy or mercapto;

(C₁-C₃)alkoxy or (C₁-C₃)alkylthio;

phenoxy or phenylthio;

benzyloxy, methylenedioxy, ethylenedioxy;

halogen;

trifluoromethyl;

nitro;

cyano (—CN);

carboxyl, esterified or protected carboxyl;

amino, mono- or di-(C₁-C₃)alkylamino, or protected amino;

(C₁-C₃)alkylcarbonyl- or (C₁-C₃)alkylcarbonylamino-;

—CONH(C₁-C₃)alkyl or —CON[(C₁-C₃)alkyl][(C₁-C₃)alkyl]; and

—NH—C(═NR₇)R₈ wherein R₇ is hydrogen, (C₁-C₃)alkyl, or an N-protecting group and R₈ is amino, mono- or di-(C₁-C₆)alkylamino, protected amino, or (C₁-C₃)alkyl.

As used herein the term “protecting group” when used in relation to an amino or carboxylic acid moeity in the compounds of this invention means a group which is used to render the amino or carboxylic acid moeity substantially non reactive, ie to neutralise its amino or carboxylic acid functionality. In this context, protected amino groups include amido and acylamino, protected hydroxy or mercapto groups include ethers and thioethers, protected carboxyl groups include esters, and imidazolyl, indolyl or guanidyl groups may be protected as t-butoxycarbonyl derivatives. These are only examples of the many protecting derivatives known in the art and the others known to a skilled person. Such protecting groups are of course well known, eg from the art of peptide synthesis, and are discussed in the widely used handbook by T. W. Greene and P. G. M. Wuts, Protective groups in Organic Synthesis, 2nd Edition, Wiley, New York 1991, and elsewhere in the chemical literature.

In accordance with the preferred embodiment of the second aspect of the present invention there is provided a derivatives of dihydropyrimidines of formula I

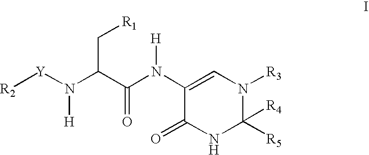

or a pharmaceutical acceptable salt, hydrate or solvate thereof

Wherein:

Y is selected from —C(O)—, —OC(O)—, or —S(O₂)_;

R₁ is selected from isopropyl, cyclohexyl, phenyl, tert-butylphenyl, isopropylphenyl, 4-fluorophenyl, 4-methoxyphenyl, 3-pyridinyl, naphthyl, biphenyl, 3,4-methylenedioxy-phenyl, benzothienyl, quinolinyl, isoquinolinyl, tetrahydroquinolinyl, tetrahydronaphthyl; aminonaphthyl; or acetamidonaphthyl.

R₂ is selected from 2-fluoroethyl, cyclohexyl, phenyl, benzyloxyphenyl, t-butylphenyl, biphenyl, benzyl, phenethyl, guanidinobenzyl, amidinobenzyl, guanidinophenethyl, amidinophenethyl, benzyloxyphenyl, naphthyl, naphthylmethyl, naphthylethyl, morpholinophenyl, morpholinobenzyl, morpholinophenethyl, 4-(2-carboxy-2-amino ethyl)-phenyl, 4-(2-carboxy-2-amino ethyl)-phenethyl, 3-pyridyl-phenyl, 3-pyridyl-phenethyl, 3-tetrazolyl-phenyl; 3,4-methylenedioxy-phenyl; 3,4-ethylenedioxy-phenyl; tetrahydroquinolinyt; dihydroquinoiinyl; benzothiophen-2-yl; 5-cloro-benzothiophen-2-yl; benzothiophen-2-yi-methyl, quinoline-2-yl; quinoline-2-yl-methyl, benzofuran-2-yl; 5-chloro-benzofuran-2-yl or benzofuran-2-yl-methyl.

R₃ is selected from hydrogen, methyl, ethyl, 2-fluoroethyl, methoxy, ethoxy, cyclopropyl,

R₄ and R₅ individually is selected from hydrogen, methyl, 2-fluoroethyl, t-butyl, t-butylmethyl, phenyl, fluorophenyl, cyclopentyl, cyclohexyl, pyridyl, carboxyphenyl, methylphenyl or furanyl.

R₄ and R₅ together are selected from oxo, cyclopentyl or cyclohexyl.

The derivative of formula I having asymmetric carbon atoms represents both R and S diastereoisomers.

The derivative of formula I having double bonds represents both E and Z geometrical isomers.

Pharmaceutically acceptable salts of the compounds of formula (I) are selected from sodium, potassium, magnesium or calcium salt of carboxylic group and hydrogen chloride, tartaric acid, succinic acid, fumaric acid, methanesulfonic acid, p-toluenesulfonic acid salt of amino group.

More specifically, the most preferred embodiments of the present invention include the following compounds:

5-[2-(Benzyloxy carbonyl)-amino-2-isobutyl]-acetamido-2-phenyl-2,3-dihydopyrimidine-6-(1H)one.

5-[2-(Benzyloxy carbonyl)-amino-2-isobutyl]-acetamido-2-(2-fluorophenyl)-2,3-dihydopyrimidine-6-(1H)one.

5-[2-(Benzyloxy carbonyl)-amino-2-isobutyl]-acetamido-2-(3-fluorophenyl)-2,3-dihydopyrimidine-6-(1H)one

5-[2-(Benzyloxy carbonyl)-amino-2-isobutyl]-acetamido-2-(4-fluorophenyl)-2,3-dihydopyrimidine-6-(1H)one

5-[2-(Benzyloxy carbonyl)-amino-2-isobutyl]-acetamido-2-(2,4-difluorophenyl)-2,3-dihydopyrimidine-6-(1H)one

5-[2-(Benzyloxy carbonyl)-amino-2-isobutyl]-acetamido-2-benzyl-2,3-dihydopyrimidine-6-(1H)one.

5-[2-(Benzyloxy carbonyl)-amino-2-isobutyl]-acetamido-2,2-spirocyclopentyl-2,3-dihydopyrimidine-6-(1H)one

5-[2-(Benzyloxy carbonyl)-amino-2-isobutyl]-acetamido-2,2-spirocyclohexyl-2,3-dihydopyrimidine-6-(1H)one

5-[2-(Benzyloxy carbonyl)-amino-2-isobutyl]-acetamido-2,2-spirocyclohexyl-2,3-dihydopyrimidine-6-(1H)one

5-[2-(Benzyloxy carbonyl)-amino-2-isobutyl 1]-acetamido-2-(pyridin-4-yl)-2,3-dihydopyrimidine-6-(1H)one

5-[2-(Benzyloxy carbonyl)-amino-2-isobutyl]-acetamido-2-(2-carboxyphenyl)-2,3-dihydopyrimidine-6-(1H)one.

5-[2-(Benzyloxy carbonyl)-amino-2-isobutyl]-acetamido-2-(4-carboxyphenyl)-2,3-dihydopyrimidine-6-(1H)one.

5-[2-(Benzyloxy carbonyl)-amino-2-isobutyl]-acetamido-2-methyl-2,3-dihydopyrimidine-6-(1H)one

5-[2-(Benzyloxy carbonyl)-amino-2-isobutyl]-acetamido-2,2-dimethyl-2,3-dihydopyrimidine-6-(1H)one

5-[2-(Benzyloxy carbonyl)-amino-2-isobutyl]-acetamido-2-tert-butylmethyl-2,3-dihydopyrimidine-6-(1H)one

5-[2-(Benzyloxy carbonyl)-amino-2-benzyl]-acetamidopyrimidin-2,6-dione.

5-[2-(Benzyloxy carbonyl)-amino-2-isobutyl]-acetamidopyrimidin-2,6-dione.

5-[2-(Benzyloxy carbonyl)-amino-2-cyclohexylmethyl]-acetamidopyrimidin-2,6-dione.

5-[2-(Benzyloxy carbonyl)-amino-2-isobutyl]-acetamido-2-(2,4-dimethylphenyl)-2,3-dihydopyrimidine-6-(1H)one

5-[2-(3-Phenylpropionyl)-amino-2-isobutyl]-acetamido-2,2-spirocyclopentyl-2,3-dihydopyrimidine-6-(1H)one

5-[2-(3-Phenylpropionyl)-amino-2-isobutyl]-acetamido-2-(furan-2-yl)-2,3-dihydopyrimidine-6-(1H)one

5-[2-(3-Phenylpropionyl)-amino-2-isobutyl]-acetamido-2,2-spirocycloheptyl-2,3-dihydopyrimidine-6-(1H)one.

5-[2-(Benzyloxy carbonyl)-amino-2-isobutyl]-acetamido-2-(furan-2-yl)-2,3-dihydopyrimidine-6-(1H)one

5-[2-(Benzothiophene-2-yl)-amino-2-isobutyl]-acetamido-2-(2-fluorophenyl)-2,3-dihydopyrimidine-6-(1H)one

5-[2-(4-Benzyloxybenzoyl)-amino-2-isobutyl]-acetamido-2-(furan-2-yl)-2,3-dihydopyrimidine-6-(1H)one

In accordance with the preferred embodiment of the third aspect of the present invention there is provided a derivatives of dihydropyrimidines of formula I

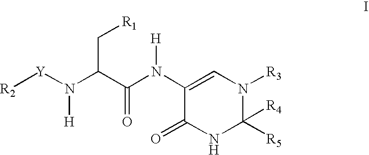

or a pharmaceutical acceptable salt, hydrate or solvate thereof

Wherein:

Y is selected from —C(O)—;

R₁ is isopropyl, cyclohexyl and phenyl.

R₂ is t-butylphenyl, biphenyl, phenethyl, morpholinoethyl, benzothiophen-2-yl or benzofuran-2-yl.

R₃ is selected from hydrogen or methyl,

R₄ and R₅ individually is fluorophenyl, pyridyl, or furanyl.

R₄ and R₅ together is cyclopentyl or cyclohexyl.

The derivative of formula I having asymmetric carbon atoms represents both R and S diastereoisomers.

The derivative of formula I having double bonds represents both E and Z geometrical isomers.

Pharmaceutically acceptable salts of the compounds of formula (I) is sodium salt of carboxylic acid and hydrogen chloride salt of amino group.

As stated, the compounds of the invention are inhibitors of cysteine proteases, for example cathepsins B, L and S or K. The invention therefore also provides a pharmaceutical composition containing a compound of formula (I) as defined above, and a pharmaceutically acceptable carrier. Also provided is the use of such a compound in the preparation of a composition for inhibiting cysteine protease activity in the body of a mammal suffering a disease mediated by such activity, and a method of treatment of an animal suffering from a disease mediated by cysteine protease activity, which method comprises administering to the mammal a sufficient amount of a compound of formula (I) as defined above to inhibit such activity.

Diseases mediated by cysteine protease activity include muscular dystrophy, osteoporosis, tumour metastasis, rheumatoid arthritis, neuronal or cardiac ischaemia, allergic immune response, and protozoal or bacterial disease.

Compositions with which the invention is concerned may be prepared for administration by any route consistent with the pharmacokinetic properties of the active ingredient(s).

Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

For topical application to the skin, the active ingredient(s) may be made up into a cream, lotion or ointment. Cream or ointment formulations, which may be used for the drug are conventional formulations well known in the art, for example as described in standard textbooks of pharmaceutics such as the British Pharmacopoeia.

The active ingredient(s) may also be administered parenterally in a sterile medium. Depending on the vehicle and concentration used, the drug can either be suspended or dissolved in the vehicle. Advantageously, adjuvants such as local anaesthetic, preservative and buffering agents can be dissolved in the vehicle. Intravenous infusion is another route of administration for the compounds.

Safe and effective dosages for different classes of patient and for different disease states will be determined by clinical trial as is required in the art. It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

### DESCRIPTION OF THE PREFERRED EMBODIMENTS

The present invention provides certain novel derivatives of dihydropyrimidine having excellent cysteine protease inhibitory activity particularity to cathepsins. The compounds of this invention are characterized by having a substitution at position 2,3, and 5 of dihydropyrimidin-6-one.

Compounds of general formula I, reported in the present invention are prepared by the reaction of compound (II) with various aldehydes or ketones in appropriate solvents, which fall within the art of chemistry, as shown in the Scheme-1.

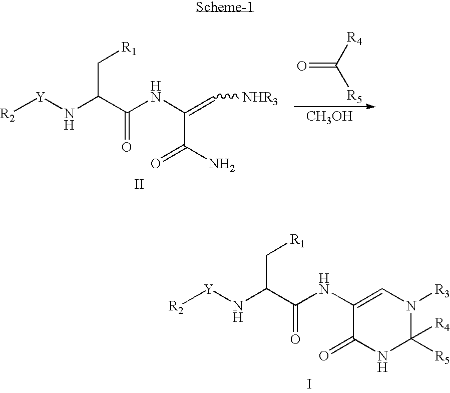

Alternatively, the derivatives of general formula I was also prepared by the general synthetic route as represented in scheme II

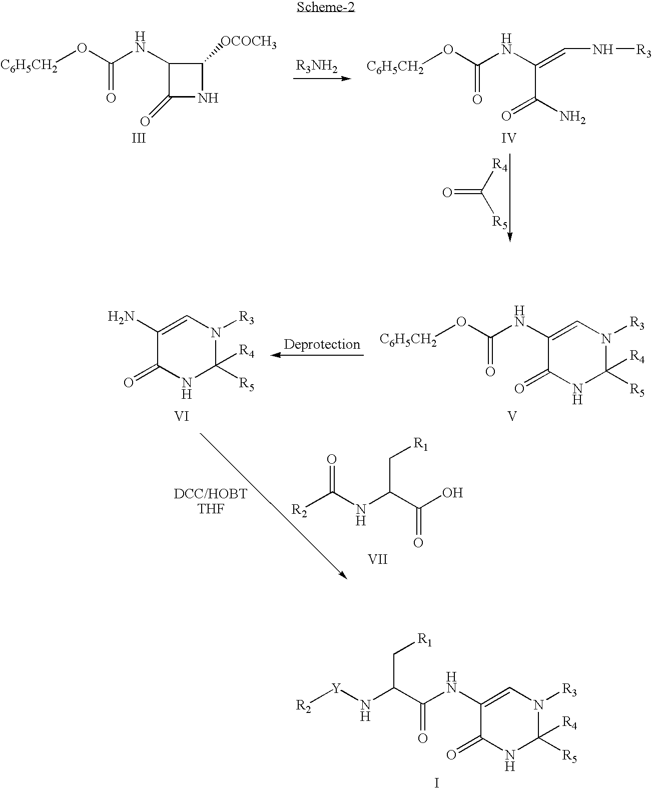

The compound III was treated with amines followed by a reaction with substituted aldehyde or ketones gave protected compound V. The benzyloxycarbonyl protected compound V was deprotected by hydrogenation in the presence of a metal catalyst, such as Pd, Pt, or Rh, under normal pressure to high pressure to give compound VI. Further, compound VI was reacted with substituted carboxylic acid VII in the presence of DCC, or with acid chlorides in the presence of base, or with anhydride in the presence of base or the activated ester, gave compound I.

The preparation of compound II was carried out by the synthetic procedure as described in Scheme-3.

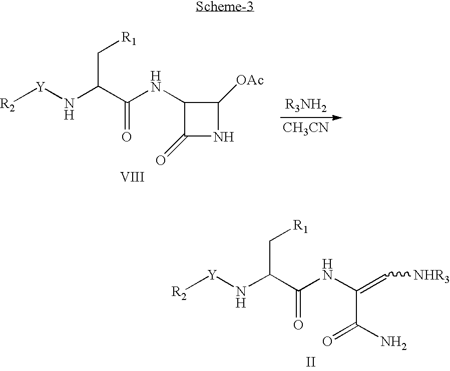

Alternatively compound II can also be prepared by following the synthetic scheme-4.

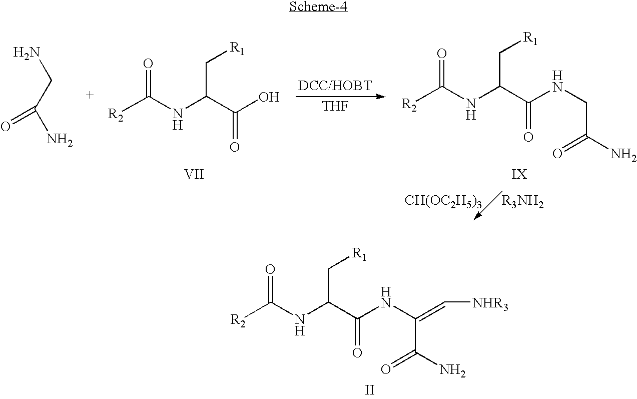

The compound VII residue is defined as substitution at position-5 of 5-amino-dihydropyrimidin-6-one. The compound VII was coupled with aminoacetamide either in the presence of DCC, or with its acid chloride in the presence of base, or with its anhydride in the presence of base or with its activated ester.

In the above processes, the reactants are reacted together with solvent at elevated or low temperatures for sufficient time to allow the reaction to proceed to completion. The reaction conditions will depend upon the nature and reactivity of the reactants. Wherever a base is used in a reaction, it is selected from the group consisting of triethyl amine, pyridine, 4-dimethylaminopyridine, diisopropylamine, 1,5-diazabicyclo [4,3,0] non-5-ene, 1,8-diazabicyclo [5,4,0] undec-7-ene, sodium carbonate, potassium carbonate, cesium carbonate, sodium hydroxide, potassium hydroxide. Depending on the reactants, a solvent will generally be selected from the group consisting of benzene, toluene, acetonitrile, tetrahydrofuran, ethanol, methanol, chloroform, ethyl acetate, methylene chloride, dimethyl formamide, dimethyl sulfoxide, hexamethyl phosphoric triamide, water, pyridine, acetone and the like solvent mixtures may also be utilized. Reaction temperatures generally range from between −70° C. to 150° C. The preferred molar ratio of reactants is 1:1 to 5. The reaction time range from 0.5 to 72 hours, depending on the reactants.

The following examples illustrate embodiments of the invention.

### EXAMPLE-1

### NPI-3243

5-[2-(Benzyloxy carbonyl)amino-2-isobutyl]acetamido-2-phenyl-2,3-dihydopyrimidine-6-(1H)one.

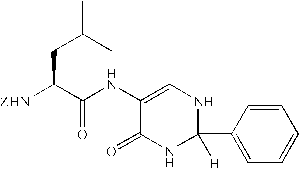

A solution of 2-(2S-2-benzyloxycarbonylamino-2-isopropylmethyl-acetamido)-3-amino-acrylamide (1.78 g) in methanol (100 ml) was treated with benzaldehyde (5 g) and refluxed for 10 h. The solvent was removed in vacuo and the residue was purified by silica gel column chromatography using a mixture of chloroform and methanol (5%) as to give the title compound.

Yield: 0.268 g, 12%

m.p.: 76-78

¹HNMR(DMSO-d₆): δ 0.85 (m, 6H), 1.40-1.70 (m, 3H), 4.00-4.20 (m, 1H), 5.05 (s, 2H), 5.62 (s, 1H), 7.06 (d, 1H, J=6.0 Hz), 7.20-7.50 (m, 10H), 7.60-7.70 (m, 2H), 7.78 (s, 1H), 8.43 (s, 1H).

### EXAMPLE-2

### NPI-3392

5-[2-(Benzyloxy carbonyl)amino-2-isobutyl]acetamido-2-(2-fluorophenyl)-2,3-dihydopyrimidine-6-(1H)one.

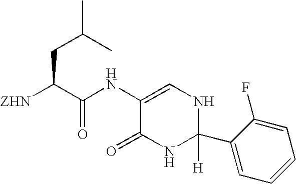

The above title compound was synthesized by the procedure described for step-2 of example-1 and using 2-(2S-2-benzyloxycarbonylamino-2-isopropylmethyl-acetamido)-3-amino-acrylamide and 2-fluorobanzaldehyde.

Yield: 8%

m.p.: 78-81° C.

¹HNMR(DMSO-d₆): δ 0.83-0.89 (m, 6H), 1.44-1.70 (m, 3H), 4.06-4.18 (m, 1H), 5.05 (s, 2H), 5.91 (s, 1H), 7.01-7.67 (m, 12H), 7.79 (s, 1H), 8.43 (s, 1H).

### EXAMPLE-3

### NPI-3474

5-[2-(Benzyloxy carbonyl)amino-2-isobutyl]acetamido-2-(3-fluorophenyl)-2,3-dihydopyrimidine-6-(1H)one

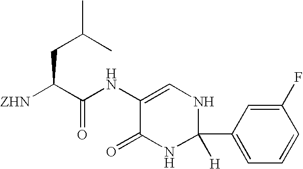

The title compound was synthesized by the procedure described for step-2 of example-1 and using 2-(2S-2-benzyloxycarbonylamino-2-isopropylmethyl-acetamido)-3-amino-acrylamide and 3-fluorobanzaldehyde.

Yield: 17%

m.p.: 84-86° C.

¹HNMR(DMSO-d₆): δ 0.82-0.88(m, 6H), 1.41-1.73(m, 3H), 4.02-4.15(m, 1H), 5.04(s, 2H), 5.66(s, 1H), 7.19-7.70(m, 12H), 7.97(s, 1H), 8.39(s, 1H).

### EXAMPLE-4

### NPI-3470

5-[2-(Benzyloxy carbonyl)amino-2-isobutyl]acetamido-2-(4-fluorophenyl)-2,3-dihydopyrimidine-6-(1H)one

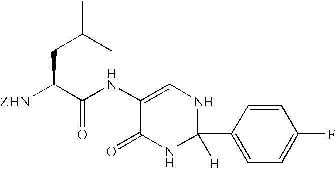

The above title compound was synthesized by the procedure described for step-2 of example-1 and using 2-(2S-2-benzyloxycarbonylamino-2-isopropylmethyl-acetamido)-3-amino-acrylamide and 4-fluorobanzaldehyde.

Yield: 23%

m.p.: 87-91° C.

¹HNMR(DMSO-d₆): δ 0.82-0.88 (m, 6H), 1.33-1.74 (m, 3H), 4.02-4.17 (m, 1H), 5.05 (s, 2H), 5.64 (s, 1H), 7.07-7.72 (m, 12H), 7.87 (s, 1H), 8.39 (s, 1H).

### EXAMPLE-5

### NPI-3490

5-[2-(Benzyloxy carbonyl)-amino-2-isobutyl]acetamido-2-(2,4-difluorophenyl)-2,3-dihydopyrimidine-6-(1H)one

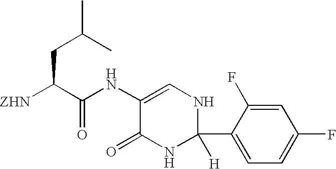

The title compound was synthesized by the procedure described for step-2 of example-1 and using 2-(2S-2-benzyloxycarbonylamino-2-isopropylmethyl-acetamido)-3-amino-acrylamide and 2,4-difluorobanzaldehyde.

Yield: 9%

m.p.: 87-90° C.

¹HNMR(DMSO-d₆): δ 0.83-0.89 (m, 6H), 1.45-1.74 (m, 3H), 4.03-4.16 (m, 1H), 5.05 (s, 2H), 5.88 (s, 1H), 7.00-7.35 (m, 8H), 7.59-7.69 (m, 2H), 7.81 (s, 1H), 8.44 (s, 1H).

### EXAMPLE-6

### NPI-3469

5-[2-(Benzyloxy carbonyl)amino-2-isobutyl]acetamido-2,2-spirocyclopentyl-2,3-dihydopyrimidine-6-(1H)one

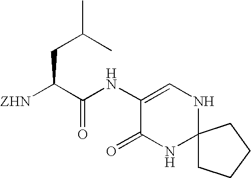

The above title compound was synthesized by the procedure described for step-2 of example-1 and using the 2-(2S-2-benzyloxycarbonylamino-2-isopropylmethyl-acetamido)-3-amino-acrylamide and cyclopentanone.

Yield: 3.4%

m.p.: 80-82° C.

¹HNMR(DMSO-d₆): δ 0.80-0.88 (m, 6H, 1.41-1.92(m, 11H), 4.02-4.15 (m, 2H), 5.05 (s, 2H), 6.80 (d, 1H, J=6.0 Hz), 7.35 (s, 5H), 7.56 (d, 1H, J=6.0 Hz), 7.58 (s, 1H), 7.67 (d, 1H, J=9.0 Hz), 8.32 (s, 1H).

### EXAMPLE-7

### NPI-3481

5-[2-(Benzyloxy carbonyl)-amino-2-isobutyl]acetamido-2,2-spirocyclohexyl-2,3-dihydopyrimidine-6-(1H)one.

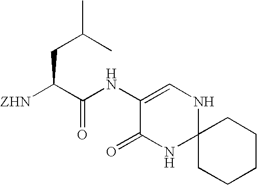

The above title compound was synthesized by the procedure described for step-2 of example-1 and using 2-(2S-2-benzyloxycarbonylamino-2-isopropylmethyl-acetamido)-3-amino-acrylamide and cyclohexanone.

Yield: 4.5%

m.p.: 83-85° C.

¹HNMR(DMSO-d₆): δ 0.83-0.88 (m, 6H), 1.15-1.94 (m, 13H), 3.90-4.18 (m, 2H), 5.02 and 5.05 (2s, 2H), 6.68 (d, 1H, J=6.0 Hz), 7.35 (s, 6H), 7.54 (d, 1H, J=6.0 Hz), 7.66 (d, 1H, J=8.3 Hz), 8.31 (s, 1H).

### EXAMPLE-8

### NPI-3479

5-[2-(Benzyloxy carbonyl)-amino-2-isobutyl]acetamido-2-tert-butyl-2,3-dihydopyrimidine-6-(1H)one

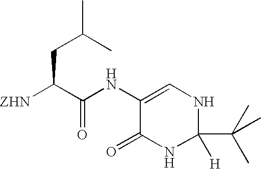

The above title compound was synthesized by the procedure described for step-2 of example-1 and using 2-(2S-2-benzyloxycarbonylamino-2-isopropylmethyl-acetamido)-3-amino-acrylamide and trimethyl acetaldehyde.

Yield: 4.5%

m.p.: 87-88° C.

¹HNMR(DMSO-d₆): δ 0.83-0.90(m, 15H), 1.40-1.72 (m, 3H), 3.99-4.15 (m, 1H), 4.20 (s, 1H), 5.05 (s, 2H), 6.54 (d, 1H, J=5.0 Hz), 7.25-7.71 (m, 8H), 8.35(d, 1H, J=2.5 Hz).

### EXAMPLE-9

### NPI-3468

5-[2-(Benzyloxy carbonyl)-amino-2-isobutyl]acetamido-2-(pyridin-4-yl)-2,3-dihydopyrimidine-6-(1H)one

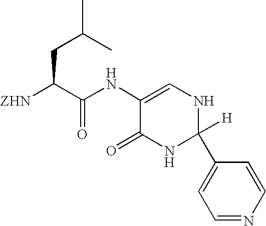

The above title compound was synthesized by the procedure described for step-2 of example-1 and using 2-(2S-2-benzyloxycarbonylamino-2-isopropylmethyl-acetamido)-3-amino-acrylamide and pyridine-4-carboxaldehyde.

Yield: 20.4%

m.p.: 97-99° C.

¹HNMR(DMSO-d₆): δ 0.84-0.88 (m, 6H), 1.44-1.71 (m, 3H), 3.90-4.00 (m, 1H), 5.01 (s, 2H), 5.47 (d, 1H, J=8.1 Hz), 6.37 (br, s, 1H), 6.67-6.75 (m, 2H), 7.36 (s, 6H), 7.60-7.65 (m, 1H), 8.59 (s, 1H), 8.62 (s, 1H), 8.71-8.90 (m, 2H).

EXAMPLE-10

### NPI-3400

5-[2-(Benzyloxy carbonyl)-amino-2-isobutyl]acetamido-2-tert-butylmethyl-2,3-dihydopyrimidine-6-(1H)one

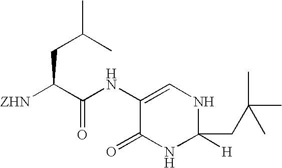

The above title compound was synthesized by the procedure described for step-2 of example-1 and using 2-(2S-2-benzyloxycarbonylamino-2-isopropylmethyl-acetamido)-3-amino-acrylamide and 3,3-dimethylbutyraldehyde.

Yield: 13%

m.p.: 84-86° C.

¹HNMR(DMSO-d₆): δ 0.83-0.98 (m, 15H), 1.45-1.64 (m, 5H), 4.05-4.15 (m, 1H), 4.54 (s, 1H), 5.05 (s, 2H), 6.45 (d, 1H, J=4.3 Hz), 7.26-7.35 (m, 7H), 7.62-7.72 (m, 2H), 8.35 (m, 1H).

### EXAMPLE-11

### NPI-3398

5-[2-(Benzyloxy carbonyl)-amino-2-isobutyl]acetamido-2-(2-carboxyphenyl)-2,3-dihydopyrimidine-6-(1H)one.

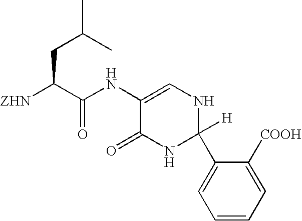

The title compound was synthesized by the procedure described for step-2 of example-1 and using 2-(2S-2-benzyloxycarbonylamino-2-isopropylmethyl-acetamido)-3-amino-acrylamide and 3-carboxy benzaldehyde.

Yield: 3%

m.p.: 221-223° C.

¹HNMR(DMSO-d₆): δ 0.82-0.92 (m, 6H), 1.43-1.76 (m, 3H), 4.05-4.17 (m, 1H), 5.05 (s, 2H), 6.39 (s, 1H), 6.80-6.88 (m, 1H), 7.36-7.67 (m, 10H), 7.96 (d, 1H, J=7.6 Hz), 8.41 (s, 1H), 13.35 (s, 1H).

EXAMPLE-12

### NPI-3397

5-[2-(Benzyloxy carbonyl)-amino-2-isobutyl]acetamido-2-(4-carboxyphenyl)-2,3-dihydopyrimidine-6-(1H)one.

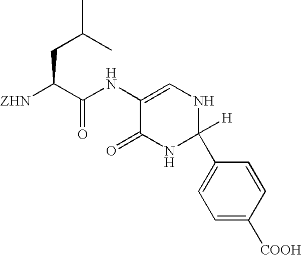

The above title compound was synthesized by the procedure described for step-2 of example-1 and using 2-(2S-2-benzyloxycarbonylamino-2-isopropylmethyl-acetamido)-3-amino-acrylamide and 4-carboxy benzaldehyde.

Yield: 13%

m.p.: 139-145° C.

¹HNMR(DMSO-d₆): δ 0.82-0.88(m, 6H), 1.35-1.80 (m, 3H), 3.98-4.18 (m, 1H), 5.04 (s, 2H), 5.71 (s, 1H), 7.20-7.98 (m, 13H), 8.39 (s, 1H), 13.04 (s, 1H).

### EXAMPLE-13

### NPI-3267

5-[2-(Benzyloxy carbonyl)amino-2-benzyl]acetamido pyrimidin-2,6-dione

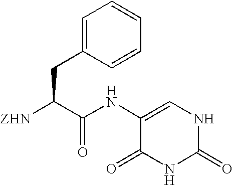

A mixture of N-benzyloxycarbonylamino-phenylalanine (0.429 g 1.433 mmol), DCC (0.296 g, 1.433 mmol), 1-HBT (0.194 g, 1.433 mmol) and 5 amino uracil (0.182 g, 1.433 mmol) in dry DMF (10 ml) was stirred at RT for 6 hrs and diluted with ethyl acetate. The ethyl acetate solution was washed with water, aq. sat. NaHCO₃ solution followed by brine solution, dried over MgSO₄, filtered and evaporated. The crude product obtained was treated with isopropanol and the solid separated was filtered and dried to give the title compound.

Yield: 90.1%

m.p.: 229-230° C.

¹HNMR(DMSO-d₆): δ 2.76-3.06 (m, 2H), 4.50-4.60 (m, 1H), 4.94 (s, 2H), 7.23-7.40 (m, 10H), 7.72 (d, 1H, J=8.4 Hz), 8.10 (s, 1H), 9.35 (s, 1H), 11.15 (br, s, 2H).

### EXAMPLE-14

### NPI-3268

5-[2-(Benzyloxy carbonyl)-amino-2-isobutyl]acetamido Pyrimidin-2,6-dione.

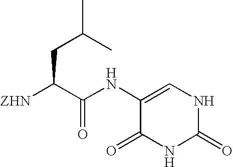

The above compound was prepared by the procedure described in example-13 and by using N-(benzyloxycarbonyl)amino leucine (0.511 g, 1.93 mmol), DCC (0.397 g, 1.93 mmol), 1-HBT (0.261 g, 1.93 mmol) and 5-amino uracil (0.245, 1.93 mmol) in DMF (12 ml).

Yield: 52.4%

m.p.: 205-206° C.

¹HNMR(DMSO-d₆): δ 0.84-0.89 (m, 6H), 1.35-1.70 (m, 3H), 4.22-4.32 (m, 1H), 5.04 (s, 2H), 7.35 (s, 5H), 7.70 (d, 1H, J=8.1 Hz), 8.06 (s, 1H), 9.06 (s, 1H), 10.67 (br, s, 1H), 11.50 (br, s, 1H).

EXAMPLE-15

### NPI-3269

5-[2-(Benzyloxy carbonyl)-amino-2-cyclohexylmethyl]-acetamido pyrimidin-2,6-dione

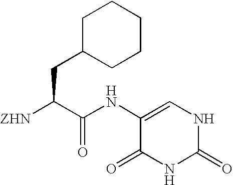

The above compound was prepared by the procedure described in example-13 and by using N-(benzyloxycarbonyl)amino cyclohexylalanine (0.505 g, 1.654 mmol), DCC (0.341 g, 1.654 mmol), 1-HBT (0.224 g, 1.654 mmol) and 5-amino uracil (0.105, 1.654 mmol) in DMF (15 ml).

Yield: 45%

m.p.: 204-205° C.

¹HNMR(DMSO-d₆): δ 0.74-1.76(m, 13H), 4.25-4.35(m, 1H-), 5.05(s, 2H), 7.35(s, 5H), 7.69(d, 1H, J=8.0 Hz), 8.05(s, 1H), 9.04(s, 1H), 10.66(br, s, 1H), 11.51 (br s, 2H).

### Example-16

### NPI-3497

5-[2-(Benzyloxy carbonyl)-amino-2-isobutyl]acetamido-2-(2,4-dimethylphenyl)-2,3-dihydopyrimidine-6-(1H)one

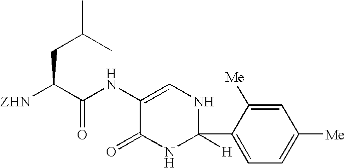

The above title compound was synthesized by the procedure described for step-2 of example-1 and using 2-(2S-2-benzyloxycarbonylamino-2-isopropylmethyl-acetamido)-3-amino-acrylamide and 2,4-dimethyl benzaldehyde.

Yield: 5.1%

m.p.: 101-106° C.

¹HNMR(DMSO-d₆): δ 0.84-0.89(m, 6H), 1.45-1.70 (m, 3H), 2.27 and 2.34 (2s, 6H), 4.06-4.20 (m, 1H), 5.06 (s, 2H), 5.76 (s, 1H), 6.79 (d, 1H, J=5.0 Hz), 7.03-7.42 (m, 9H), 7.56 (s, 1H), 7.71 (d, 1H, J=6.2 Hz), 8.42 (s, 1H).

### EXAMPLE-17

### NPI-4769

5-[2-(3-Phenylpropionyl)-amino-2-isobutyl]acetamido-2,2-spirocyclopentyl-2,3-dihydopyrimidine-6-(1H)one

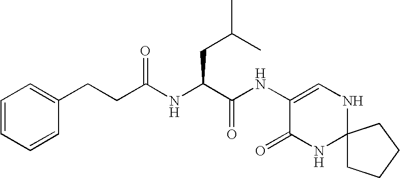

The above title compound was synthesized by the procedure described for step-2 of example-1 and using 2-[2S-2-(3-phenylpropionoyl)-amino-2-isopropylmethyl-acetamido]-3-amino-acrylamide and cyclopentanone.

Yield: 3.4%

m.p.: 95° C.

¹HNMR(DMSO-d₆): δ 0.78-0.87(m, 6H), 1.39-1.94(m, 11H), 2.43-2.50(m, 2H), 2.80-2.90(m, 2H), 4.24-4.37(m, 1H), 6.82(d, 1H, J=6.5 Hz), 7.13-7.33(m, 6H), 7.54(d, 1H, J=6.5 Hz), 8.17(d, 1H, J=6.5 Hz), 8.28(s, 1H).

### EXAMPLE-18

### NPI-4772

5-[2-(3-Phenylpropionyl)-amino-2-isobutyl]acetamido-2-(furan-2-yl)-2,3-dihydopyrimidine-6-(1H)one

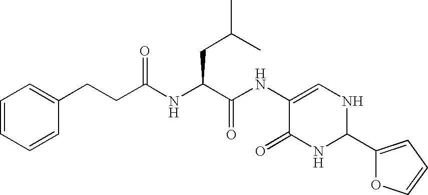

The above title compound was synthesized by the procedure described for step-2 of example-1 and using 2-(2S-2-(3-phenylpropionoyl)-amino-2-isopropylmethyl-acetamido)-3-amino-acrylamide and furan-2-carboxaldehyde.

Yield: 4.4%

m.p.: 124° C.

¹HNMR(DMSO-d₆): δ 0.79-0.90 (m, 6H), 1.40-1.50 (m, 3H), 2.40-2.50 (m, 2H), 2.77-2.88 (m, 2H), 4.27-4.40 (m, 1H), 5.67 (s, 1H), 6.35-6.47 (m, 2H), 7.10-7.30 (m, 7H), 7.60 (d, 1H, J=5.83 Hz), 7.66 (s, 1H), 7.91 (s, 1H), 8.16 (d, 1H, J=8.5 Hz), 8.35 (s, 1H).

### EXAMPLE-19

### NPI-4774

5-[2-(3-Phenylpropionoyl)-amino-2-isobutyl]acetamido-2,2-spirocycloheptyl-2,3-dihydopyrimidine-6-(1H)one.

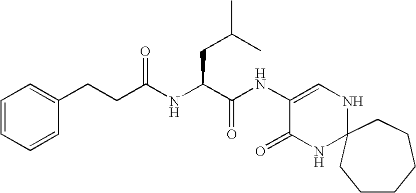

The above title compound was synthesized by the procedure described for step-2 of example-1 and using 2-(2S-2-(3-phenylpropionoyl)-amino)-2-isopropylmethyl-acetamido)-3-amino-acrylamide and cycloheptanone.

Yield: 6.3%

m.p.: 125° C.

¹HNMR(DMSO-d₆): δ 0.80-0.90 (m, 6H), 1.30-1.62 (m, 10H), 1.74-2.00 (m, 3H), 2.39-2.48 (m, 2H), 2.78-2.89 (m, 2H), 4.25-4.41 (m, 1H), 6.74 (d, 1H, J=6.3 Hz), 7.10-7.51 (m, 7H), 8.14 (d, 1H, J=8.3 Hz).

### EXAMPLE-20

### NPI-3510

5-[2-(Benzyloxy carbonyl)amino-2-isobutyl]acetamido-2-(furan-2-yl)-2,3-dihydopyrimidine-6-(1H)one

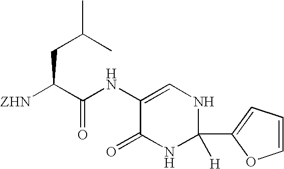

The above title compound was synthesized by the procedure described for step-2 of example-1 and using 2-(2S-2-benzyloxycarbonylamino-2-isopropylmethyl-acetamido)-3-amino-acrylamide and furan-2-carboxaldehyde.

Yield: 4.8%

m.p.: 75-80° C.

¹HNMR(DMSO-d₆): δ 0.85-0.90 (m, 6H), 1.42-1.72 (m, 3H), 4.03-4.17 (m, 1H), 5.05 (s, 2H), 5.68 (s, 1H), 6.36 (s, 1H), 6.43 (s, 1H), 7.10 (s, 1H), 7.35 (s, 6H), 7.66 (s, 2H), 7.92 (s, 1H), 8.38 (s, 1H).

### EXAMPLE-21

### NPI-3493

5-[2-(Benzothiophene-2-yl)amino-2-isobutyl]acetamido-2-(2-fluorophenyl)-2,3-dihydopyrimidine-6-(1H)one

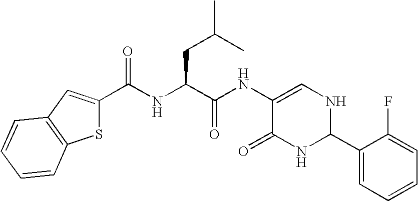

The above title compound was synthesized by the procedure described for step-2 of example-1 and using 2-(2S-2-benzothein-2yl-carbonylamino-2-isopropylmethyl-acetamido)-3-amino-acrylamide and 2-fluorobenzaldehyde.

Yield: 11.3%

m.p.: 119-123° C.

¹HNMR(DMSO-d₆): δ 0.90-0.94 (m, 6H), 1.58-1.85 (m, 3H), 4.56-4.68 (m, 1H), 5.91 (s, 1H), 7.08 (d, 1H, J=5.0 Hz), 7.20-7.65 (m, 8H), 7.75 (s, 1H), 7.97-8.09 (m, 2H), 8.27 (s, 1H), 8.57 (s, 1H), 8.93 (d, 1H, J=8.4 Hz).

### EXAMPLE-22

### NPI-3522

5-[2-(4-Benzyloxybenzoyl)amino-2-isobutyl]acetamido-2-(furan-2-yl)-2,3-dihydopyrimidine-6-(1H)one

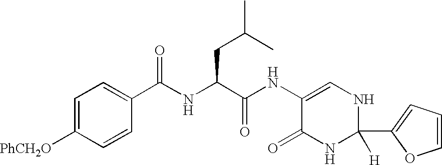

The above title compound was synthesized by the procedure described for step-2 of example-1 and using 2-[2S-2-(4-benzyloxybenzoylamino)-2-isopropylmethyl-acetamido]-3-amino-acrylamide and furan-2-carboxaldehyde.

Yield: 28%

m.p.: 103-105° C.

¹HNMR(DMSO-d₆): δ 0.90-0.97 (m, 6H), 1.54-1.79 (m, 3H), 4.50-4.62 (m, 1H), 5.19 (s, 2H), 5.66 (s, 1H), 6.35-6.44 (m, 2H), 7.08-7.12 (m, 3H), 7.33-7.49 (m, 6H), 7.67 (s, 1H), 7.86-7.90 (m, 3H), 8.40 (s, 1H), 8.50 (d, 1H, J=6.5 Hz).

### Biological Testing

Testing of inhibitors for inhibition of cathepsin B, L, K and S.

In Vitro Assay Procedure for Cathepsin B

The compounds of formula I were tested for inhibition of cathepsin B using the known method (A. J. Barret et al., Biochem. J. 1982, 201, 189-198). To a 170 μl of enzyme-buffer mixture (enzyme: r rat cathepsin B, diluted to give approximate 10 F units/min, buffer: 56 mM sodium acetate, 1.124 mM EDTA, 10 mM DTT, pH 5.1) a 10 μl of inhibitor (dissolved in DMSO) was added. After 10 min of incubation at room temperature, a 20 μl of 5 mM substrate (N-CBZ-Phe-Arg-AMC, dissolved in DMSO) was added to initiate reaction. Reading is followed up for 10 min at the fluoroscan reader (excitation at 380 nm emission at 460 nm).

A plot of percentage of inhibition vs inhibitor concentration is obtained, and IC₅₀ is determined using a linear regression calculations (concentration of inhibitor which will give 50% inhibition).

In Vitro Assay Procedure for Cathepsin L

To a 170 μl of enzyme-buffer mixture (enzyme: r rat cathepsin L, diluted to give approximate 15 F units/min, buffer: 58.8 mM sodium citrate, 1.18 mM EDTA, 235 mM sodium chloride, 5 mM DTT, pH 5.0) a 10 of inhibitor (dissolved in DMSO) was added. After 10 min of incubation at room temperature, a 20 μl of 1 mM substrate (N-CBZ-Phe-Arg-AMC, dissolved in DMSO) was added to initiate reaction. Reading is followed up for 10 min at the fluoroscan reader (excitation at 380 nm emission at 460 nm).

A plot of percentage of inhibition vs inhibitor concentration is obtained, and IC₅₀ is determined using a linear regression calculations (concentration of inhibitor which will give 50% inhibition).

In Vitro Assay Procedure for Cathepsin K

To a 170% of enzyme-buffer mixture (enzyme: r cathepsin K, diluted to give approximate 30 F units/min, buffer: 100 mM sodium acetate, 5 mM EDTA, 20 mM L-cysteine, 0.01% Brij, pH 5.5) a 10 μl of inhibitor (dissolved in DMSO) was added. After 10 min of incubation at room temperature, a 20 μl of 2.7 mM substrate (N-CBZ-Phe-Arg-AMC, dissolved in DMSO) was added to initiate reaction. Reading is followed up for 10 min at the fluoroscan II plate reader (excitation at 380 nm emission at 460 nm).

A plot of percentage of inhibition vs inhibitor concentration is obtained, and IC₅₀ is determined using a linear regression calculations (concentration of inhibitor which will give 50% inhibition).

In Vitro Assay Procedure for Cathepsin S

To a 170% of enzyme-buffer mixture (enzyme: r cathepsin S, diluted to give approximate 30 F units/min, buffer: 100 mM sodium phosphate, 1 mM EDTA, 5 mM DTT, 0.01% Brij, pH 6.5) a 10 μl of inhibitor (dissolved in DMSO) was added. After 10 min of incubation at room temperature a 201 μl of 1.2 mM substrate (N-CBZ-Val-Val-Arg-AMC, dissolved in DMSO) was added to initiate reaction. Reading is followed up for 10 min at the fluoroscan 11 plate reader (excitation at 380 nm emission at 460 nm).

A plot of percentage of inhibition vs inhibitor concentration is obtained, and IC₅₀ is determined using a linear regression calculations (concentration of inhibitor which will give 50% inhibition).

TABLE 1
In vitro inhibitory activity of compounds with Cathepsins

| | IC50 (μM) | | | |
|---|---|---|---|---|
| Example No. | Cathepsin B | Cathepsin L | Cathepsin K | Cathepsin S |
| 1 | 4.01 | 0.46 | 0.34 | 0.46 |
| 2 | 8.38 | 0.92 | 0.057 | 0.29 |
| 6 | 3.16 | 1.79 | 0.1 | 0.48 |
| 8 | 4.56 | 0.48 | 0.074 | 0.48 |
| 9 | 2.29 | 0.46 | 0.018 | 0.055 |
| 10 | 9.53 | 2.32 | 0.25 | 2.32 |
| 14 | >50 | >50 | >50 | >50 |
| 17 | >50 | >50 | 12.12 | 8.49 |
| 19 | 5.22 | 0.45 | 0.091 | 0.068 |
| 20 | 2.35 | 1.5 | 0.045 | 0.094 |
| 21 | 0.42 | 0.17 | 0.014 | 0.037 |

Selected compounds of present invention were tested in rodents. This class of compound has favorable pharmacokinetics at the oral dose of 5 mg/kg. The bioavailability is about 60-70%. The data is summarized in Table-2.

TABLE 2
Pharmacokinetic parameters of selected examples with mice after
single oral dose of 5 mg/kg

| PK parameters | Example #6 | Example #21 |
|---|---|---|
| Cmax (ug/ml) | 3.83 ± 3.09 | 2.61 ± 1.84 |
| AUC (ug.min/ml) | 163.6 ± 21.25 | 51.20 ± 15.82 |
| T1/2 (hr) | 1.63 ± 0.48 | 0.54 ± 0.39 |
| Cl/F (ml/mm) | 0.617 ± 0.08 | 2.09 ± 0.72 |
| Vz/F (L/kg) | 4.49 ± 1.9 | 4.49 ± 3.03 |

1. In accordance with the present invention, there is provided novel dihydropyrimidine derivatives of general formula (I): 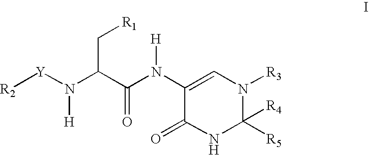
Wherein:
Y represents —C(O)—, —OC(O)—, —NHC(O)— or —S(O₂)—;
R₁ represents hydrogen or an optionally substituted C₁-C₆alkyl, C₂-C₆alkenyl, C₂-C₆alkynyl, cycloalkyl, cycloalkenyl, aryl or heterocyclic group.
R₂ represents hydrogen or an optionally substituted C₁-C₆alkyl, C₂-C₆alkenyl, C₂-C₆alkynyl, cycloalkyl, cycloalkenyl, aryl or heterocyclic group.
R₃ represents H, R₆ and OR₆, wherein R₆ is C₁-C₃alkyl, C₂-C₃alkenyl, C₂-C₃alkynyl, cycloalkyl, cycloalkenyl, aryl or a heterocyclic group.
R₄ and R₅ individually represent H or an optionally substituted C₁-C₆alkyl, C₂-C₆alkenyl, C₂-C₆alkynyl, cycloalkyl, cycloalkenyl, aryl, or heterocyclic group.
R₄ and R₅ together represents an oxo group or a C₃-C₆ cyclic ring system, which may be further, substituted with hydroxyl, halogen, and amino and substituted amino groups.
or a pharmaceutically acceptable salt, hydrate or solvate thereof

2. A compound according to claim 1 wherein the unqualified term “substituted” as applied to a group means substituted with 1, 2, or 3 substituents selected from
(C₁-C₃)alkyl;
phenyl;
C₃-C₆cycloalkyl;
heterocyclic;
hydroxy or mercapto;
(C₁-C₃)alkoxy or (C₁-C₃)alkylthio;
phenoxy or phenylthio;
benzyloxy, methylenedioxy, ethylenedioxy;
halogen;
trifluoromethyl;
nitro;
cyano (—CN);
carboxyl, esterified or protected carboxyl;
amino, mono- or di-(C₁-C₃)alkylamino, or protected amino;
(C₁-C₃)alkylcarbonyl- or (C₁-C₃)alkylcarbonylamino-;
—CONH(C₁-C₃)alkyl or —CON[(C₁-C₃)alkyl] [(C₁-C₃)alkyl]; and
—NH—C(═NR₇)R₈ wherein R₇ is hydrogen, (C₁-C₃)alkyl, or an N-protecting group and R₈ is amino, mono- or di-(C₁-C₆)alkylamino, protected amino, or (C₁-C₃)alkyl.

3. A compound according to claim 1 wherein the term “(C₁-C₆) alkyl” or “lower alkyl” means a straight or branched chain alkyl moiety having from 1 to 6 carbon atoms, including for example, methyl, ethyl, propyl, 1-methylethyl, butyl, 1-methylpropyl, 2-methylprop-1-yl, 2-methylprop-2-yl, pentyl, 3-methylbutyl, and hexyl. Similar terms such as “(C₁-C₃) alkyl” are to be interpreted similarly.

4. A compound according to claim 1 wherein the term “C₂-C₆alkenyl” means a straight or branched chain alkenyl moiety having from 2 to 6 carbon atoms having at least one double bond, for example, vinyl, allyl, 1- and 2-butenyl and 2-methyl-2-propenyl. Similar terms such as “(C₂-C₃)alkenyl” are to be interpreted similarly.

5. A compound according to claim 1 wherein the term “C₂-C₆ alkynyl” means a straight chain or branched chain hydrocarbon groups having from two to six carbon atoms and having in addition one triple bond, for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl. Similar terms such as “(C₂-C₃)alkynyl” are to be interpreted similarly.

6. A compound according to claim 1 wherein the term “cycloalkyl” means a saturated alicyclic moiety having from 3-7 carbon atoms and includes, for example, cyclohexyl, cycloheptyl, cyclopentyl, cyclobutyl and cyclopropyl.

7. A compound according to claim 1 wherein the term “aryl” refers to a mono-, bi- or tri-cyclic, substituted or unsubstituted, carbocyclic aromatic group, and to groups consisting of two covalently linked substituted or unsubstituted monocyclic carbocyclic aromatic groups, for example phenyl, biphenyl and napthyl, tetrahydronaphthyl, dihydronaphthyl, and cyclohexyl phenyl.

8. A compound according to claim 1 wherein the unqualified term “heterocyclic” means a 5-7 membered heterocyclic ring, which may be aromatic or non-aromatic, containing one or more heteroatoms selected from S, N and O, and optionally fused to a benzene or hetero-atom containing ring, for examples 1,2,3-triazolyl, 1,2,4-triazolyl, 1,2,3,4-tetrazolyl, thienyl, furyl, pyrrolyl, imidazolyl, pyridyl, pyrimidinyl, oxazolyl, thiazolyl, thiadiazolyl, pyrazolyl, pyrrolidinyl, morpholinyl, thiomorpholinyl, piperazinyl, piperidinyl, benzofuranyl, benzothiophenyl, benzimidazolyl, quinolinyl, isoquinolinyl, indolyl, tetrahydroquinolinyl, tetrahydroisoquinolinyl, pyridylphenyl and pyrimidylphenyl groups.

9. In accordance with the preferred embodiment of the second aspect of the present invention there is provided a derivatives of 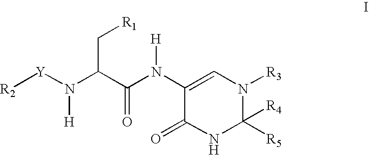
dihydropyrimidines of formula I
Wherein:
Y is selected from —C(O)—, —OC(O)—, or —S(O₂)_;
R₁ is selected from isopropyl, cyclohexyl, phenyl, tert-butylphenyl, isopropylphenyl, 4-fluorophenyl, 4-methoxyphenyl, 3-pyridinyl naphthyl, biphenyl, 3,4-methylenedioxy-phenyl, benzothienyl, quinolinyl, isoquinolinyl, tetrahydroquinolinyl, tetrahydronaphthyl; aminonaphthyl; or acetamidonaphthyl.
R₂ is selected from 2-fluoroethyl, cyclohexyl, phenyl, benzyloxyphenyl, t-butylphenyl, biphenyl, benzyl, phenethyl, guanidinobenzyl, amidinobenzyl, guanidinophenethyl, amidinophenethyl, benzyloxyphenyl, naphthyl, naphthylmethyl, naphthylethyl, morpholinophenyl, morpholinobenzyl, morpholinophenethyl, 4-(2-carboxy-2-amino ethyl)-phenyl, 4-(2-carboxy-2-amino ethyl)-phenethyl, 3-pyridyl-phenyl, 3-pyridyl-phenethyl, 3-tetrazolyl-phenyl; 3,4-methylenedioxy-phenyl; 3,4-ethylenedioxy-phenyl; tetrahydroquinolinyl; dihydroquinolinyl; benzothiophen-2-yl; 5-cloro-benzothiophen-2-yl; benzothiophen-2-yl-methyl, quinoline-2-yl; quinoline-2-yl-methyl, benzofuran-2-yl; 5-chloro-benzofuran-2-yl or benzofuran-2-yl-methyl.
R₃ is selected from hydrogen, methyl, ethyl, 2-fluoroethyl, methoxy, ethoxy, cyclopropyl, R₄ and R₅ individually is selected from hydrogen, methyl, 2-fluoroethyl, t-butyl, t-butylmethyl, phenyl, fluorophenyl, cyclopentyl, cyclohexyl, pyridyl, carboxyphenyl, methylphenyl or furanyl.
R₄ and R₅ together are selected from oxo, cyclopentyl or cyclohexyl.
or a pharmaceutically acceptable salt, hydrate or solvate thereof

10. In accordance with the preferred embodiment of the third aspect of the present invention there is provided a derivatives of dihydropyrimidines of formula I 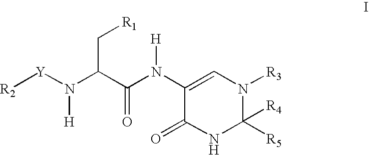
Wherein:
Y is selected from —C(O)—;
R₁ is isopropyl, cyclohexyl and phenyl.
R₂ is t-butylphenyl, biphenyl, phenethyl, morpholinoethyl, benzothiophen-2-yl or benzofuran-2-yl.
R₃ is selected from hydrogen or methyl,
R₄ and R₅ individually is fluorophenyl, pyridyl, or furanyl.
R₄ and R₅ together is cyclopentyl or cyclohexyl.
or a pharmaceutical acceptable salt, hydrate or solvate thereof

11. As used herein the term “halogen” means fluoro, chloro, bromo or iodo

12. A compound according to claim 1 wherein the derivative of formula I having asymmetric carbon atoms represents both R and S diastereoisomers.

13. A compound according to claim 1 wherein the derivative of formula I having double bonds represents both E and Z geometrical isomers.

14. A compound according to claim 1 wherein pharmaceutically acceptable salts of the compounds of formula (I) are selected from sodium, potassium, magnesium or calcium salt of carboxylic group and hydrogen chloride, tartaric acid, succinic acid, fumaric acid, methanesulfonic acid, p-toluenesulfonic acid salt of amino group.

15. A pharmaceutical composition containing a compound as claimed in any of the preceding claims and a pharmaceutically acceptable carrier.

16. The use of a compound as claimed in any of claims 1 to 14 in the preparation of a composition for inhibiting cysteine protease activity particularly cathepsins in the body of a mammal suffering a disease mediated by such activity.

17. A method of treatment of an animal suffering from a disease mediated by cysteine protease activity, which method comprises administering to the mammal a sufficient amount of a compound as claimed in any of claims 1 to 14 to inhibit such activity.

18. The use as claimed in claim 16 or a method as claimed in claim 17 wherein the disease is muscular dystrophy, osteoporosis, tumour metastasis, rheumatoid arthritis, neuronal or cardiac ischaemia, allergic immune response, and protozoal or bacterial diseases.
